(19) United States
(12) Patent Application Publication
(10) Pub. No.: US 2023/0218641 A1
(43) Pub. Date: Jul. 13, 2023

(54) ANTI-ACNE COMPOSITIONS

(71) Applicant: L'OREAL, Paris (FR)

(72) Inventors: Lisa BECKERMANN, Chembur Mumbai (IN); Suresh CHAWRAI, Chembur Mumbai (IN); Yogesh SURADKAR, Chembur Mumbai (IN)

(21) Appl. No.: 17/999,220

(22) PCT Filed: May 19, 2021
(86) PCT No.: PCT/EP2021/063321

§ 371 Date: Nov. 18, 2022

(30) Foreign Application Priority Data
May 19, 2020 (IN) 202011021118
Jul. 7, 2020 (FR) FR20 07162

(51) Int. Cl. A61K 31/60; A61K 47/38; A61K 47/32; A61K 47/18; A61K 47/10; A61K 47/06; A61K 47/36; A61K 31/455; A61K 33/30; A61K 31/201; A61K 31/4412

(52) U.S. Cl. CPC A61K 31/60; A61K 47/38; A61K 47/32; A61K 47/183; A61K 47/10; A61K 47/06; A61K 47/36; A61K 31/455; A61K 33/30; A61K 31/201; A61K 31/4412

(57) ABSTRACT

The present invention relates to a composition, preferably a cosmetic composition, comprising, in an aqueous phase:
at least one anti-acne active agent; and
hydroxyethylcellulose and at least poly(2-acrylamido 2-methyl propane sulfonic acid),
   and uses thereof.

The present invention relates to cosmetic compositions that are useful for personal skin care, especially for anti-acne skin care.

Skin disorders, such as acne, can be irritating to the skin and embarrassing to the person suffering from the disorder. This is the most common reason for a visit to a dermatologist. There are many treatments, but no cure for acne. These include antibiotics (which inhibit the growth of Propionibacterium acnes bacteria which play a role in acne), retinoids such as Roaccutane® or Differin® (which reduce sebaceous gland output of sebum), and antimicrobials such as benzoyl peroxide, α-hydroxy acids or β-hydroxy acids. Acne lesions result from the rupture of a sebaceous follicle, followed by inflammation and pus (a “whitehead”), or by accumulation of plugged material in the sebaceous follicle (a “blackhead”). It is thus important to keep the skin clean, and provide the lesions with active agents. Unfortunately, cleansing is not always sufficient. Furthermore, the active agents used for the treatment of acne tend to be harsh and irritating. Furthermore, the more powerful actives may need to be applied by a dermatologist.

Among the different compositions available for treating acne, one can quote anti-acne leave-on daily facial care gels, which usually present a low pH, i.e. of around 3.5 to 4.5. Indeed, the maintenance of such a low pH is important for this kind of product, so that it is effective against acne.

However, formulating a cosmetic product at this pH range is difficult, notably because the choice of polymers is highly limited, as most of the polymers gain viscosity and display suitable rheological properties at a pH of at least 5.5.

There is thus a need to formulate a composition for caring for the skin, which has an anti-acne effect, which has a low pH, which is stable, non-sticky, non-noodling; and which provides additional benefits such as excellent application and spreading, and contributes to the overall acceptable sensorial properties of the final product at a low pH.

The Applicant has now discovered that it is possible to formulate such compositions having the desired properties as described above.

Specifically, the Applicant has discovered that it is possible to formulate compositions for caring for and/or making up of keratin materials, which are stable, non-sticky, and which show good consistency and spreading. Said compositions comprise a unique combination of at least two specific polymers, which enable physical viscoelastic properties in acceptable ranges for good consistency and spreading.

Accordingly, the present invention relates to a composition, preferably a cosmetic composition, comprising, in an aqueous phase:
at least one anti-acne active agent; and
hydroxyethylcellulose and at least poly(2-acrylamido 2-methyl propane sulfonic acid).

The composition of the invention is an aqueous gel. It is stable, non-sticky and non-noodling. Besides, it is quickly absorbed when applied onto skin, and shows a fluidic gel texture. Moreover, the composition of the invention is efficient on acne treatment, preferably in reducing non-inflammatory lesions and/or inflammatory lesions and/or skin sebum level.

By “stable”, it is meant that the composition of the invention does not show any sedimentation (i.e. collection of the fatty globules at the bottom of the container) or dephasing (i.e. separation of the aqueous and fatty phases) over time, especially during one month, preferably during two months, under different temperatures (4° C., 37° C. and 45° C.).

By “noodling effect”, it is meant that the formulation or the polymer (hydroxyethylcellulose and/or 2-acrylamido 2-methyl propane sulfonic acid polymer) comes off with normal rubbing. This results in non-acceptance by the consumers.

Other subjects and characteristics, aspects and advantages of the invention will emerge even more clearly on reading the description and the examples that follows.

In that which follows and unless otherwise indicated, the limits of a range of values are included within this range, in particular in the expressions “of between” and “ranging from . . . to . . . ”.

Moreover, the expression “at least one” used in the present description is equivalent to the expression “one or more”.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

Viscosity of the Composition of the Invention

The composition of the invention shows a viscosity of from 1500 mPa·s to 2300 mPa·s, preferably from 1600 mPa·s to 2200 mPa·s, preferably from 1700 mPa·s to 2100 mPa·s.

Viscosity is measured according to the following protocol:

Viscosity is measured at room temperature, with the mobile M3 of Rheometer Lamy Rheology RM 200, at least 24 h after preparation of the composition.

Anti-Acne Agent

The composition comprises at least one anti-acne agent.

The term “anti-acne agent” especially means any active agent that has effects on the specific flora of greasy skin, for instance Propionibacterium acnes (P. acnes).

These effects may be bactericidal.
The anti-acne agent may be chosen from:
salicylic acid and its derivatives, such as salts and esters;
niacinamide, niacin, and nicotinic acid esters;
peroxides, including benzoyl peroxide, stabilized hydrogen peroxide and peroxides of organic acids, such as a lauroyl peroxide;
metal gluconate, such as zinc gluconate, copper gluconate or their mixtures;
asiatic acid,
the monoethanolamine salt of 1-hydroxy-4-methyl 6-trimethylpentyl-2-pyridone (INCI name: piroctone olamine), marketed especially under the trademark Octopirox® by Clariant,
citronellic acid, perillic acid (or 4-isopropenylcyclohex-1-enecarboxylic acid), glyceryl 2-ethylhexyl ether (INCI name: ethylhexylglycerine), for example marketed under the trademark Sensiva SC 50® by Schulke & Mayr,
glyceryl caprylate/caprate, for example marketed under the trademark Capmul MCM® by Abitec;
sodium calcium phosphosilicate, especially marketed under the trademarks Bioactive Glasspowder® and Actysse Premier BC® by Schott Glass;
silver-based particles, for example those marketed under the trademark Metashine ME 2025 PS® by Nippon Sheet Glass;
hop cone extract (Humulus lupulus) obtained by supercritical CO₂ extraction, such as the product marketed under the trademark HOP CO2-TO Extract® by Flavex Naturextrakte,
St. John's Wort extract obtained by supercritical CO₂ extraction, such as the product marketed under the trademark St. John's Wort CO2-TO Extract® by Flavex Naturextrakte, the mixture of extracts of roots of Scutellaria baicalensis, of Paeonia suffruticosa and Glycyrrhiza glabra, such as the product marketed under the trademark BMB-CF® by Naturogin,
argan tree extract, for instance Argapure L59710® from Cognis;
bearberry leaf extracts, for instance the product marketed under the trademark Melfade-J by Pentapharm;
10-hydroxy-2-decanoic acid such as Acnacidol P® from Vincience, sodium ursolate, azelaic acid, diiodomethyl p-tolyl sulfone such as Amical Flowable® from Angus, malachite powder, zinc oxide such as Zincare® from Elementis GMBH, octadecenedioic acid such as Arlatone dioic DCA® from Uniqema; ellagic acid; 2,4,4′-trichloro-2′-hydroxydiphenyl ether (or triclosan), 1-(3′,4′-dichlorophenyl)-3-(4′-chlorophenyl)urea (or triclocarban), 3,4,4′-trichlorocarbanilide, 3′,4′,5′-trichlorosalicylanilide, phenoxyethanol, phenoxypropanol, phenoxyisopropanol, hexamidine isethionate, metronidazole and salts thereof, miconazole and salts thereof, itraconazole, terconazole, econazole, ketoconazole, saperconazole, fluconazole, clotrimazole, butoconazole, oxiconazole, sulfaconazole, sulconazole, terbinafine, ciclopirox, ciclopiroxolamine, undecylenic acid and salts thereof, 3-hydroxybenzoic acid, 4-hydroxybenzoic acid, phytic acid, N-acetyl-L-cysteine, lipoic acid, arachidonic acid, resorcinol, 3,4,4′-trichlorocarbanalide, octoxyglycerine or octoglycerine, octanoylglycine such as Lipacid CBG® from SEPPIC, caprylyl glycol, 10-hydroxy-2-decanoic acid, dichlorophenylimidazoldioxolane and derivatives thereof described in WO 93/18743, iodopropynyl butylcarbamate, 3,7,11-trimethyldodeca-2,5,10-trienol or farnesol, phytosphingosines; quaternary ammonium salts, for instance cetyltrimethylammonium salts and cetylpyridinium salts, and
mixtures thereof.

Preferably, the anti-acne agent is chosen from salicylic acid and its derivatives, such as salts and esters; niacinamide, niacin, and nicotinic acid esters; metal gluconate, such as zinc gluconate, copper gluconate; and their mixtures. More preferably, the anti-acne agent is chosen from salicylic acid, niacinamide, zinc gluconate and their mixtures. More preferably, the anti-acne agent is a mixture of salicylic acid, niacinamide and zinc gluconate.

Preferably, the anti-acne agent(s) is present in the composition of the present invention in an amount ranging from 0.01% to 20% by weight, preferably from 0.1% to 15% by weight, more preferably from 1% to 10% by weight, more preferably from 2% to 6% by weight, relative to the total weight of the composition.

Aqueous Phase

The composition of the invention comprises an aqueous phase, which comprises hydroxyethylcellulose and at least poly(2-acrylamido 2-methyl propane sulfonic acid).

Said aqueous phase is preferably present in an amount ranging from 10% to 99% by weight, more preferably from 20% to 97% by weight of the total weight of the composition.

The composition of the invention preferably comprises water. Water is preferably present in the composition of the present invention in an amount ranging from 1% to 90% by weight, preferably from 5% to 87% by weight, more preferably from 10% to 85% by weight, relative to the total weight of the composition.

The aqueous phase may further comprise at least one polyol. The polyols may be chosen from polyols having from 2 to 20 carbon atoms, preferably from 2 to 10 carbon atoms, and preferentially having from 2 to 6 carbon atoms, such as glycerol, propylene glycol, butylene glycol, pentylene glycol, hexylene glycol, caprylylglycol, dipropylene glycol and diethylene glycol.

The aqueous phase may further comprise at least one organic solvent miscible with water different from polyols. Said organic solvent miscible with water (at room temperature 25° C.) may be chosen from monoalcohols having from 2 to 6 carbon atoms such as ethanol, isopropanol; glycol ethers (notably having from 3 to 16 carbon atoms) such as mono-, di- or tri-propylene glycol (C₁-C₄)alkyl ethers, mono-, di- or tri-ethylene glycol (C₁-C₄) alkyl ethers and mixtures thereof.

The polyols and/or organic solvents miscible with water may be present in the composition of the present invention in an amount ranging from 1% to 30% by weight, preferably from 3% to 20% by weight, more preferably from 4% to 10% by weight, relative to the total weight of the composition.

The aqueous phase may also comprise sodium hyaluronate. Said ingredient may be beneficial for skin hydration. Preferably, sodium hyaluronate is present in the composition of the present invention in an amount ranging from 0.01% to 10% by weight, preferably from 0,015% to 5% by weight, more preferably from 0.1% to 1% by weight, relative to the total weight of the composition.

Hydroxyethylcellulose

The composition of the invention comprises at least hydroxyethylcellulose (HEC).

HEC is a cellulose-derived polymer, and is a hydrophilic thickening agent.

HEC according to the invention is generally present in amounts of active material ranging from 0.01 to 20% by weight, more preferably from 0.1 to 10% by weight, even more preferably from 0.15 to 5% by weight and more particularly from 0.2 to 3% by weight relative to the total weight of the composition.

AMPS® Homopolymer (Poly(2-Acrylamido 2-Methyl Propane Sulfonic Acid))

The composition of the invention comprises at least one crosslinked or non-crosslinked homopolymer comprising 2-acrylamido 2-methyl propane sulfonic acid units (AMPS®).

The 2-acrylamido 2-methyl propane sulfonic acid homopolymer may be crosslinked or non-crosslinked.

They are water-soluble, water-dispersible or water-swellable copolymers.

Preferably, the AMPS® homopolymers used in accordance with the invention may be partially or completely neutralized with an inorganic base (such as sodium hydroxide, potassium hydroxide or aqueous ammonia) or an organic base such as mono-, di- or triethanolamine, an aminomethylpropanediol, N-methylglucamine or basic amino acids such as arginine and lysine, and mixtures of these compounds. They are generally neutralized. In the present invention, the term “neutralized” is intended to mean polymers that have been completely or almost completely neutralized, i.e. at least 90% neutralized.

The AMPS® homopolymers used in the composition of the invention generally have a number-average molecular weight ranging from 1000 to 20 000 000 g/mol, preferably ranging from 20 000 to 5 000 000, and even more preferably from 100 000 to 1 500 000 g/mol.

When the homopolymers are crosslinked, the crosslinking agents may be chosen from compounds with an olefinic polyunsaturation commonly used for crosslinking polymers obtained by radical polymerization. As crosslinking agents, mention may, for example, be made of divinylbenzene, diallyl ether, dipropylene glycol diallyl ether, polyglycol diallyl ethers, triethylene glycol divinyl ether, hydroquinone diallyl ether, ethylene glycol di(meth)acrylate, tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allyl ethers of alcohols of the sugar series, or other allyl or vinyl ethers of polyfunctional alcohols, and also allyl esters of phosphoric acid derivatives and/or vinylphosphonic acid derivatives, or mixtures of these compounds.

The degree of crosslinking generally ranges from 0.01 to 10 mol %, and more particularly from 0.2 to 2 mol %, relative to the polymer.

A preferred polymer of the present invention is poly(2-acrylamido 2-methyl propane sulfonic acid), which is partially neutralized with ammonia and highly cross-linked, such as ammonium polyacryloyldimethyl taurate, also known under the tradename Hostacerin AMPS®, and commercially available from the supplier Clariant.

Preferably, the composition of the invention comprises poly(2-acrylamido 2-methyl propane sulfonic acid).

The AMPS® homopolymer according to the invention is generally present in amounts of active material ranging from 0.01 to 20% by weight, more preferably from 0.1 to 10% by weight, even more preferably from 0.5 to 5% by weight and more particularly from 0.8 to 3% by weight relative to the total weight of the composition.

Preferably, the AMPS® homopolymer according to the invention is present in a weight ratio of active material of (AMPS® homopolymer):(hydroxyethylcellulose) ranging from 3:1 to 30:1, preferably from 4:1 to 25:1, preferably from 4:1 to 7:1.

Surfactant

The composition of the invention may comprise at least one surfactant. This surfactant can be anionic, non-ionic, amphoteric, zwitterionic or cationic. It is generally introduced in the aqueous phase.

It can have at 25° C. a HLB balance (Hydrophilic-Lipophilic Balance) in terms of GRIFFIN, preferably greater than or equal to 8. The HLB value as per GRIFFIN is defined in J. Soc. Cosm. Chem. 1954 (volume 5), pages 249-256. Reference may be made to the document “Encyclopedia of Chemical Technology, KIRK-OTHMER”, volume 22, p. 333-432, 3rd edition, 1979, WILEY, for the definition of the properties and emulsifying functions of surfactant agents, in particular p. 347-377 of this reference.

Preferably, the surfactant according to the invention is chosen from:

a) anionic surfactants such as:
polyoxyethylenated fatty acid salts and particularly those derived from alkaline salts, and mixtures thereof;
phosphoric esters and their salts such as “DEA oleth-10 phosphate” (Crodafos N 10N from CRODA) or monopotassium monocetyl phosphate (Amphisol K from Givaudan);
sulfosuccinates such as “Disodium PEG-5 citrate lauryl sulfosuccinate” and “Disodium ricinoleamido MEA sulfosuccinate”;
alkylethersulfates such as sodium lauryl ether sulfate;
isethionates;
acylglutamates such as “Disodium hydrogenated tallow glutamate” (AMISOFT HS-21 R® marketed by AJINOMOTO) and sodium stearoyl glutamate (AMISOFT HS-11 PF® marketed by AJINOMOTO) and mixtures thereof;
derivatives of soybeans such as potassium soyate;
citrates, such as Glyceryl stearate citrate (Axol C 62 Pellets from Degussa);
derivatives of proline, such as Sodium palmitoyl proline (Sepicalm VG from Seppic), or the Mixture of Sodium palmitoyl sarcosinate, Magnesium palmitoyl glutamate, palmitic acid and Palmitoyl proline (Sepifeel One from Seppic);
lactylates, such as Sodium stearoyl lactylate (Akoline SL from Karlshamns AB);
sarcosinates, such as sodium palmitoyl sarcosinate (Nikkol sarcosinate PN) or the mixture of Stearoyl sarcosine and Myristoyl sarcosine 75/25 (Crodasin SM from Croda);
sulfonates, such as Sodium C14-17 alkyl sec sulfonate (Hostapur SAS 60 from Clariant);
glycinates, such as sodium cocoyl glycinate (Amilite GCS-12 from Ajinomoto).
C16-C30 fatty acid salts in particular those derived from amines, such as triethanolamine stearate and/or amino-2-methyl-2-propane di-of-1,3 stearate;

b) amphoteric or zwitterionic surfactants, such as N-acyl-aminoacids such as N-alkyl-aminoacetates (such as trimethylglycine), disodium cocoamphodiacetate, amine oxides such as stearamine oxide or even silicone surfactants such as dimethicone copolyol phosphates such as the one sold under the trade name PECOSIL PS 100® by PHOENIX CHEMICAL;

c) non-ionic surfactants with a HLB greater than or equal to 8 at 25° C., such as:
esters and ethers of oses such as the mixture of cetylstearyl glucoside and cetyl and stearyl alcohols such as Montanov 68 from Seppic;
oxyethylene and/or oxypropylene ethers (that may comprise from 1 to 150 oxyethylene and/or oxypropylene groups) of glycerol;
oxyethylene and/or oxypropylene ethers (that may comprise from 1 to 150 oxyethylene and/or oxypropylene groups) of fatty alcohols (particularly C8-C24 and preferably C12-C18 alcohols) such as oxyethylene ether of cetearylic alcohol with 30 oxyethylene groups (CTFA name “Ceteareth-30”), oxyethylene ether of stearylic alcohol with 20 oxyethylene groups (CTFA name “Steareth-20”), and oxyethylene ether of the mix of C12-C15 fatty alcohols containing 7 oxyethylene groups (CTFA name “C12-15 Pareth-7”) marketed under the name NEODOL 25-7® by SHELL CHEMICALS,
fatty acid esters (in particular C8-C24 acid, and preferably C16-C22) and polyethylene glycol (able to comprise from 1 to 150 ethyleneglycol patterns) such as PEG-50 stearate and PEG-40 monostearate sold under the trade name MYRJ 52P® by ICI UNIQUEMA,
fatty acid esters (particularly C8-C24 acid, and preferably C16-C22 acid) and oxyethylenated and/or oxypropylated glycerol ethers (that may include 1 to 150 oxyethylanated and/or oxypropylenated groups), such as PEG-200 glyceryl monostearate sold particularly under the name Simulsol 220 TM® by SEPPIC; polyethoxylated glyceryl stearate with 30 ethylene oxide groups such as the TAGAT S® product sold by GOLDSCHMIDT, polyethoxylated glyceryl oleate with 30 ethylene oxide groups like the TAGAT O® product sold by GOLDSCHMIDT, polyethoxylated glyceryl cocoate with 30 ethylene oxide groups like the VARIONIC LI 13® product sold by SHEREX, polyethoxylated glyceryl isostearate with 30 ethylene oxide groups such as the TAGAT L® product sold by GOLDSCHMIDT and polyethoxylated glyceryl laurate with 30 groups of ethylene oxide like the TAGAT I® product from GOLDSCHMIDT,
fatty acid esters (particularly C8-C24 acid and preferably C16-C22 acid) and oxyethylenated and/or oxypropylenated sorbitol ethers (possibly containing 1 to 150 oxyethylenated and/or oxypropylenated groups), such as polysorbate 20 sold under the name Tween 20® by CRODA, polysorbate 60 sold under the name Tween 60® by CRODA,
oxyalkylenated (oxyethylenated and/or oxypropylenated) fatty acid esters, for instance the mixture PEG-100 stearate/glyceryl stearate sold, for example, by the company Croda under the name Arlacel 165;
polydimethylsiloxanes comprise both oxyethylenated groups and oxypropylene groups, such as dimethicone copolyol with INCI name PEG/PPG-17/18 DIMETHICONE, such as that sold under the trade name Q2-5220 Resin Modifier® by DOW CORNING,
dimethicone copolyol benzoate (FINSOLV SLB 101® and 201® from FINTEX),
copolymers of propylene oxide and of ethylene oxide (also called EO/PO polycondensates), and more particularly copolymers consisting of polyethylene glycol/polypropylene glycol blocks, such as for example polyethylene glycol/polypropylene glycol/polyethylene glycol triblock polycondensates, for example those having the following chemical structure:

H—(O—CH2-CH2)a-(O—CH(CH3)-CH2)b-(O—CH2-CH2)a-OH,

in which formula a ranges from 2 to 120, and b ranges from 1 to 100.

As a EO/PO polycondensate that can be used, mention can be made of polyethylene glycol/polypropylene glycol/polyethylene glycol triblock polycondensates sold under the trade names SYNPERONIC® such as SYNPERONIC PE/L44® and SYNPERONIC PE/F127® by ICI;

d) cationic surfactants such as primary, secondary or tertiary fatty amine salts, optionally polyoxyalkylene, quaternary ammonium salts, and mixtures thereof. As quaternary ammonium salts, mention can in particular be made of those satisfying the following general formula:

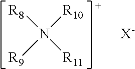

wherein:
R8 to R11, identical or different, each represent an aliphatic group, linear or branched, comprising from 1 to 30 carbon atoms, or an aromatic group such as aryl or alkylaryl, with the understanding that at least one of the R8 to R11 groups comprise from 8 to 30 carbon atoms, and preferably from 12 to 24 carbon atoms. Preferably, the R8 to R11 aliphatic groups are chosen from C1-C30 alkyl groups, C1-C30 alkoxy, polyoxyalkylene (C2-C6), C1-C30 alkylamide, alkyl(C12-C22)amidoalkyl(C2-C6), alkyl(C12-C22)acetate, and C1-C30 hydroxyalkyl; and
X— is an organic or inorganic anionic counter ion, such as the one chosen from halides, acetates, phosphates, nitrates, alkyl(C1-C4)sulfates, alkyl(C1-C4)- or alkyl(C1-C4)aryl-sulfonates, in particular methylsulfate and ethylsulfate.
   Among the quaternary ammonium salts, preference is given to tetradecyltrimethylammonium, cetyltrimethylammonium, behenyltrimethylammonium, dipalmitoylethyl-hydroxyethylmethylammonium salts, and more particularly tetradecyltrimethylammonium bromide, behenyltrimethylammonium chloride, cetyltrimethylammonium chloride or dipalmitoylethylhydroxyethylammonium methosulfate; and

e) mixtures thereof.

The surfactant may be present in an amount ranging from 0.1% to 10% by weight, preferably from 0.5% to 7% by weight, more preferably from 0.7% to 5% by weight relative to the total weight of the composition.

Fatty Phase

The composition of the invention may comprise a dispersed fatty phase.

The fatty phase may be present in an amount ranging from 0.1% to 10% by weight, preferably from 0.5% to 7% by weight, more preferably from 1% to 5% by weight relative to the total weight of the composition.

Said fatty phase preferably comprises at least one oil. The oil can be volatile or non-volatile.

The term “oil” means a water-immiscible non-aqueous compound that is liquid at room temperature (25° C.) and at atmospheric pressure (760 mmHg).

The term “non-volatile oil” means an oil that remains on keratin materials at room temperature and atmospheric pressure for at least several hours and that especially has a vapour pressure of less than 10⁻³ mmHg (0.13 Pa). A non-volatile oil may also be defined as having an evaporation rate such that, under the conditions defined previously, the amount evaporated after 30 minutes is less than 0.07 mg/cm².

These oils may be of plant, mineral or synthetic origin.

Preferably, said oil is chosen from hydrocarbonated, silicone or fluorinated oils.

The term “hydrocarbon-based oil” or “hydrocarbonated oil” means an oil formed essentially from, or even constituted by, carbon and hydrogen atoms, and optionally O and N atomes, and free of Si and F heteroatoms. Such oil can contain alcohol, ester, ether, carboxylic acid, amine and/or amide groups.

The term “silicone oil” means an oil containing at least one silicon atom, especially containing Si—O groups.

The term “fluorinated oil” means an oil containing at least one fluorine atom,

Preferably, the oil is selected from silicone oils, hydrocarbon-based volatile oils and their mixtures.

The oil can be, for example, present in an amount ranging from 0.1% to 10% by weight, preferably from 0.5% to 7% by weight, more preferably from 1% to 5% by weight relative to the total weight of the composition.

Mention may be made, for example, of hydrocarbon-based volatile oils having from 8 to 16 carbon atoms and mixtures thereof and especially branched C8-C16 alkanes such as C8-C16 isoalkanes (also known as isoparaffins), isododecane, isodecane, isohexadecane and, for example, the oils sold under the trade names Isopar or Permethyl, C8-C16 branched esters such as isohexyl neopentanoate and mixtures thereof. Isododecane or isohexadecane are preferred.

As non-volatile oils, mention may be made of:
hydrocarbon-based oils of mineral or synthetic origin, such as linear or branched hydrocarbons, for instance liquid paraffin or its derivatives, liquid petroleum jelly, polydecenes, hydrogenated polyisobutene such as Parleam sold by the company Nippon Oil Fats, squalane of synthetic or plant origin;
hydrocarbon-based oils of plant origin based on triglycerides made up of esters of fatty acids and of glycerol, the fatty acids of which may have varied chain lengths, it being possible for the latter to be linear or branched, and saturated or unsaturated, in particular the triglycerides of a fatty acid containing in particular from 4 to 22 carbon atoms, for instance heptanoic acid triglycerides, octanoic acid triglycerides and capric/caprylic acid triglycerides, or else hydroxylated triglycerides, such as sweet almond oil, calophyllum oil, palm oil, grape seed oil, sesame oil, arara oil, rapeseed oil, sunflower oil, cottonseed oil, apricot oil, castor oil, alfalfa oil, marrow oil, blackcurrant oil, macadamia oil, muscat rose oil, hazelnut oil, coriander oil, avocado oil, jojoba oil, olive oil, cereal (maize, wheat, barley, rye) germ oil, shea butter oil; esters of fatty acids, in particular having from 4 to 22 carbon atoms, and in particular of octanoic acid, of heptanoic acid, of lanolic acid, of oleic acid, of lauric acid or of stearic acid, such as propylene glycol dioctanoate, propylene glycol monoisostearate, polyglyceryl-2 diisostearate, neopentyl glycol diheptanoate;
synthetic esters of formula R1COOR2 in which R1 represents the residue of a linear or branched higher fatty acid containing from 7 to 40 carbon atoms and R2 represents a branched hydrocarbon-based chain containing from 3 to 40 carbon atoms, for instance purcellin oil (cetostearyl octanoate), isononyl isononanoate, C12 to C15 alcohol benzoate, 2-ethylhexyl palmitate, 2-octyldodecyl stearate, 2-octyldodecyl erucate, isostearyl isostearate, 2-octyldodecyl benzoate, octanoates, decanoates or ricinoleates of alcohols or of polyalcohols, isopropyl myristate, isopropyl palmitate, butyl stearate, hexyl laurate, diisopropyl adipate, 2-ethylhexyl palmitate, 2-hexyldecyl laurate, 2-octyldecyl palmitate, 2-octyldodecyl myristate, 2-diethylhexyl succinate, diisostearyl malate, isodecyl neopentanoate, hydroxylated esters such as isostearyl lactate, octyl hydroxystearate, octyldodecyl hydroxystearate, diisostearyl malate, triisocetyl citrate, glyceryl or diglyceryl triisostearate; diethylene glycol diisononanoate; pentaerythritol esters; esters of aromatic acids and of alcohols containing from 4 to 22 carbon atoms, in particular tridecyl trimellitate;
C8-C26 higher fatty acids such as myristic acid, oleic acid, linoleic acid, linolenic acid or isostearic acid;
C8-C26 higher fatty alcohols such as oleyl alcohol, linoleyl alcohol, linolenyl alcohol, isostearyl alcohol or octyldodecanol;
synthetic ethers containing at least 7 carbon atoms;
silicone oils such as linear polydimethylsiloxanes (PDMSs) that are liquid at ambient temperature, and that are optionally phenylated, such as phenyltrimethicones, phenyltrimethylsiloxydiphenylsiloxanes, diphenyl dimethicones, diphenylmethyl-diphenyltrisiloxanes, liquid 2-phenylethyltrimethyl-siloxysilicates, optionally substituted with aliphatic and/or aromatic groups, for instance alkyl, alkoxy or phenyl groups, which are pendent and/or at the end of a silicone chain, these groups containing from 2 to 24 carbon atoms, and optionally fluorinated, or with functional groups such as hydroxyl, thiol and/or amine groups; polysiloxanes modified with fatty acids or fatty alcohols or polyoxyalkylenes, such as dimethicone copolyols or alkyl methicone copolyols; liquid fluorosilicones;
and mixtures thereof.

Crosslinked Starch

The composition of the invention may comprise at least one crosslinked starch. Said crosslinked starch is also called modified starch. Crosslinked starch may be useful for improving the sensorial and oil-absorbing properties of the composition.

Starch(es) that can be used in this invention are particularly macromolecules in the form of polymers composed of elementary patterns that are anhydroglucose units. The number of these patterns and their assembly provide a means of distinguishing amylose (linear polymer) and amylopectin (ramified polymer). The relative proportions of amylose and of amylopectin, as well as their degree of polymerization, vary according to the plant origin of the starches.

The starch molecules used in this invention may originate from a plant source such as cereals, tubercles, roots, vegetables and fruits. Thus, the starch(es) may originate from a plant source chosen from among maize, peas, potatoes, sweet potatoes, banana, barley, wheat, rice, oat, sago, tapioca and sorghum. The starch is preferably derived from potatoes.

Hydrolysates from starches mentioned above may also be used.

Starches are usually in the form of a white powder, insoluble in cold water, with an elementary particle size varying from 3 to 100 microns.

The starches used in the composition according to the invention are chemically modified by crosslinking. In particular, these reactions may be performed by cross-linking by functional agents capable of reacting with hydroxyl groups of starch molecules that will thus be bonded to each other (for example with glyceryl and/or phosphate groups).

In particular, monostarch phosphates (of the Am—O—PO—(OX)2 type), distarch phosphates (of the Am—O—PO—(OX)—O—Am type) or even tristarch phosphates (of the Am—O—PO—(O—Am)2 type) or mixes of them may be obtained by cross linking with phosphorated compounds.

In particular, X denotes alkaline metals (for example sodium or potassium), alkaline earth metals (for example calcium, magnesium), ammonia salts, amine salts like monoethanolamine, diethanolamine, triethanolamine, amino-3 propanediol-1,2 salts, ammonium salts derived from basic aminoacids like as lysine, arginine, sarcosine, ornithine, citrulline.

The phosphorated compounds may for example be sodium tripolyphosphate, sodium orthophosphate, phosphorus oxichloride or sodium trimetaphosphate.

Distarch phosphates will be in particular used, or compounds rich in distarch phosphate such as the product marketed under references PREJEL VA-70-T AGGL (gelatinized hydroxypropylated manioc distarch phosphate) or PREJEL TK1 (gelatinized manioc distarch phosphate) or PREJEL 200 (gelatinized acetylated manioc distarch phosphate) by the AVEBE Company or STRUCTURE ZEA or STRUCTURE XL by the Akzo Nobel (gelatinized hydroxypropylated maize distarch phosphate).

Preferably, the crosslinked starch is a gelatinized hydroxypropylated maize distarch phosphate.

Amphoteric starches can also be used in the invention; these amphoteric starches contain one or several anionic groups and one or several cationic groups. The anionic and cationic groups may be related to the same reactive site of the starch molecule or to different reactive sites, but they are preferably related to the same reactive site. The anionic groups may be of the carboxylic, phosphate or sulfate type, and preferably carboxylic. Cationic groups may be of the primary, secondary, tertiary of quaternary amine type.

As amphoteric starches, in particular, potato starches modified by 2-chloroethyl aminodipropionic acid. Mention can be made in particular of potato starch modified by 2-chloroethyl aminodipropionic acid neutralized with soda, marketed under the reference STRUCTURE SOLANACE by NATIONAL STARCH.

O-carboxymethylated starch designates a starch that has been modified by substitution, in the free hydroxyl groups, of a hydrogen with a carboxymethylated group —CH2COOH. It can be as such, or in the form of salt, for example an alkali metal salt.
O-carboxymethylated starches can be prepared, for example, by reacting a starch with monochloroacetic acid, or a monochloroacetic acid alkali salt (for example sodium salt).
Preferably, and O-carboxymethylated starch is used that has the form of an alkali metal salt, and more preferably, in the form of a sodium salt.
Preferably, the O-carboxymethylated starch is prepared using potato starch.
The O-carboxymethylated starch can also be partially or entirely crosslinked. Preferably, it is partially crosslinked. The crosslinking of the starch can be carried out for example by heating the starch, or by having it react with crosslinking agents such as phosphates, glycerol. Even more preferably, the O-carboxymethylated starch is a sodium salt of starch, in particular of potato, O-carboxymethylated and partially crosslinked. Such a product is for example marketed under the name PRIMOJEL by AVEBE.

The crosslinked starch may be present in the composition in a content ranging from 0.1% to 8% by weight, preferably from 0.5% to 5% by weight and preferably from 0.7% to 3% by weight in relation to the total weight of the composition.

Additional Ingredients

The composition of the invention may comprise at least one additive, such as UV filters, fragrances, preservatives, vitamins, chelatants, pH regulators and/or fillers.

A person skilled in the art can adjust the type and amount of additives present in the compositions according to the invention by means of routine operations, so that the desired cosmetic properties and stability properties for these compositions are not affected by the additives.

UV filters may be mineral, such as titanium dioxide, or organic.

The chelatant may be tetrasodium glutamate diacetate (sold under the name Dissolvine GL-47-S by AkzoNobel).

The filler may be organic or mineral. Among the mineral fillers that can be used in the compositions according to the invention, mention may be made of talc, mica, silica, kaolin or bentone. Among the organic fillers, mention may be made of polyamide powders (Nylon® Orgasol from Atochem), polyalanine and polyethylene powders, polytetrafluoroethylene (Teflon®) powders, lauroyllysine, tetrafluoroethylene polymer powders, hollow polymer microspheres, such as Expancel (Nobel Industrie), metal soaps derived from organic carboxylic acids containing from 8 to 22 carbon atoms, preferably from 12 to 18 carbon atoms, for example zinc stearate, magnesium stearate or lithium stearate, zinc laurate or magnesium myristate.

Preferably, the composition of the invention comprises an emollient, especially an emollient which has the sensory feel of dimethicone. Preferably, said emollient is Diheptyl Succinate (and) Capryloyl Glycerin/Sebacic Acid Copolymer. Thus preferably, the composition of the invention comprises Diheptyl Succinate (and) Capryloyl Glycerin/Sebacic Acid Copolymer. Said copolymer may be the one marketed under the name LexFeel™ N5 MB by Inolex. Preferably, it is present in the composition of the present invention in an amount ranging from 0.01% to 10% by weight, preferably from 0.1% to 10% by weight, more preferably from 1% to 5% by weight relative to the total weight of the composition.

pH of the Composition of the Invention

The composition of the invention presents a low pH, i.e. of less than 4.7. Preferably, the pH is between 3 and 4.7, preferably between 3.5 to 4.7.

Preferably, the composition of the invention presents a low pH, i.e. of less than 4.5. Preferably, the pH is between 3 and 4.5, preferably between 3.5 to 4.5.

Methods and Use

According to an embodiment, the present invention relates to a non-therapeutic method for treating a keratin material, comprising the step of applying the composition of the present invention to the keratin material.

Preferably the present invention relates to a method for caring for the skin, comprising the step of applying the composition of the present invention to the skin.

The present invention also relates to the use of the composition of the present invention for treating acne.

The following examples serve to illustrate the invention without, however, being limiting in nature.

### EXAMPLES

### Example 1: Preparation of a Composition According to the Present Invention and Comparative Compositions

Formula A according to the invention, and comparative compositions B to H (indicated by a star in the following tables) were prepared according to the amounts given in the table below. The amounts are given in % by weight of the total composition.

The protocol is as follows:

1/ Heat about 88% of the WATER, the TETRASODIUM GLUTAMATE DIACETATE and the SALICYLIC ACID until about 70° C. under stirring at about 200 RPM.
2/ When the temperature reaches about 70° C., add the SODIUM HYDROXIDE and wait until total disappearing of the SALICYLIC ACID powder. Maintain that temperature.
3/ Add the SODIUM HYALURONATE very slowly and give it 15 minutes to swell.
4/ Add the AMMONIUM POLYACRYLOYLDIMETHYL TAURATE slowly and give it 20 minutes to swell until the formulation becomes a totally clear gel. Adjust the speed to about 600 RPM during this step.
5/ Maintain the speed at about 600 RPM, add the HYDROXYETHYLCELLULOSE and give it 15 minutes to swell.
6/ Add the melted GLYCERYL STEARATE (and) PEG-100 STEARATE, and give it 10 minutes to disperse while maintaining the temperature.
7/ Start reducing the temperature and when it reaches 60° C., slowly add the HYDROXYPROPYL STARCH PHOSPHATE and give it 20 minutes to disperse.
8/ In a separate beaker, use a particle disperser to disperse the TITANIUM DIOXIDE into the DIPROPYLENE GLYCOL. At around 55° C., add the TITANIUM DIOXIDE mixture into the main vessel.
9/ At around 35° C., add the DIMETHICONE, and reduce the speed of the stirrer to about 400 RPM.
10/ In a separate beaker, dissolve the ZINC GLUCONATE in about 6% of the WATER. At around 35° C., add the mixture to the main vessel.
11/ In a separate beaker, dissolve the NIACINAMIDE in the about 6% of the WATER. In order to facilitate the dissolution, heating to about 30° C. can help. At around 35° C., add the mixture to the main vessel.
12/ When the temperature reaches 30° C., add the ISODODECANE.
13/ If needed adjust the pH to 4.2±0.3.
14/ In a separate beaker dissolve the MENTHOL into the PERFUME. When the preparation reaches room temperature, add the mixture to the main vessel.
15/ If needed, adjust the water quantity in the main vessel.

| | FOMULATION | | | | | | | |
|---|---|---|---|---|---|---|---|---|
| | A | | | | | | | |
| | (invention) | B* | C* | D* | F* | F* | G* | H* |
| Tetrasodium glutamate diacetate | 0.1 | 0.1 | 0.1 | 0.1 | 0.1 | 0.1 | 0.1 | 0.1 |
| (Dissolvine GL-47-S from AkzoNobel) | | | | | | | | |
| Dipropylene Glycol | 5 | 5 | 5 | 5 | 5 | 5 | 5 | 5 |
| Isododecane | 1 | 1 | 1 | 1 | 1 | 1 | 1 | 1 |
| Dimethicone (5 cst) | 1 | 1 | 1 | 1 | 1 | 1 | 1 | 1 |
| Sodium hyaluronate | 0.03 | 0.03 | 0.03 | 0.03 | 0.03 | 0.03 | 0.03 | 0.03 |
| Glyceryl stearate (and) PEG-100 | 1 | 1 | 1 | 1 | 1 | 1 | 1 | 1 |
| Stearate (Arlacel 165 from Croda) | | | | | | | | |
| Hydroxyethyl cellulose | 0.3 | — | — | 0.3 | — | — | 0.3 | — |
| AMMONIUM | 1.5 | — | 1.5 | — | 1.5 | — | — | — |
| POLYACRYLOYLDIMETHYL | | | | | | | | |
| TAURATE (Hostacerin ® | | | | | | | | |
| from Clariant) | | | | | | | | |
| Hydroxypropyl methyl cellulose | — | — | — | — | 0.3 | 1.8 | — | — |
| Carbomer (Carbopol Ultrez | — | — | — | — | — | — | 1.5 | 1.8 |
| 30 from Lubrizol) | | | | | | | | |
| Talc | 1 | 1 | 1 | 1 | 1 | 1 | 1 | 1 |
| TiO2 (water soluble) | 1 | 1 | 1 | 1 | 1 | 1 | 1 | 1 |
| Hydroxypropyl starch | 1 | 1 | 1 | 1 | 1 | 1 | 1 | 1 |
| phosphate (Structure XL) | | | | | | | | |
| Salicylic acid | 1.2 | 1.2 | 1.2 | 1.2 | 1.2 | 1.2 | 1.2 | 1.2 |
| Sodium hydroxide | 0.2 | 0.2 | 0.2 | 0.2 | 0.2 | 0.2 | 0.2 | 0.2 |
| Niacinamide | 2 | 2 | 2 | 2 | 2 | 2 | 2 | 2 |
| Zinc gluconate | 0.5 | 0.5 | 0.5 | 0.5 | 0.5 | 0.5 | 0.5 | 0.5 |
| Menthol classic | 0.03 | 0.03 | 0.03 | 0.03 | 0.03 | 0.03 | 0.03 | 0.03 |
| Perfume | 0.1 | 0.1 | 0.1 | 0.1 | 0.1 | 0.1 | 0.1 | 0.1 |
| TOTAL WATER | qsp 100 | qsp 100 | qsp 100 | qsp 100 | qsp 100 | qsp 100 | qsp 100 | qsp 100 |

For each composition, viscosity, classical stability, stress stability (stress cycle), sensory tests by a panel and sensory tests by the formulator, were measured according to the following respective protocols:

### Protocol of Measurement of the Viscosity:

### Material

### Rheometer Lamy Rheology RM 200

### Mobile M3

### Procedure

Viscosity is measured at 3 time points, t=0 s, t=30 s and t=10 minutes, with the mobile M3.
For freshly formulated products, the viscosity is measured 24 h after the formulation of the product (first experiment).
For stability samples, the viscosity is measured after the sample has reached the room temperature.
Viscosity is indicated in UD in the table below. For information, 40 UD corresponds to 1700 mPa·s; 50 UD corresponds to 2100 mPa·s; and 60 UD corresponds to 2500 mPa·s.

### Protocol of Stability, “Classical Stability”:

### Material

Four chambers at controlled temperature of 4° C., 25° C., 37° C. and 45° C.
Stability glass jars of 50 g.

### Principle

The objective of this test is to simulate the process of ageing of the formula, to see if the formula is stable over the product shelf-life. The product is kept at four different temperatures (4° C., 25° C., 37° C. and 45° C.) and the stability observations are made at two different time points (1 month and 2 months). Indeed, 2 months at 45° C. is equivalent to 3 years on the shelf under real-time conditions.

### Procedure

About 50 g of the test samples in glass jars are kept in duplicates at the respective temperatures 4° C., 25° C., 37° C. and 45° C. At the time points of 1 month and 2 months respectively, one jar is being analyzed at each temperature and different parameters are assessed such as appearance, color, perfume, odor, pH and viscosity. If the product is compliant with the target values, the product is considered as stable at the time point. The observations have to be noted down and if any deviation is not acceptable, the product is considered as not stable over time.

### Protocol of Stability, “Stress Cycle”:

### Material

Three chambers at controlled temperature of 4° C., 25° C. and 50° C.
Stability glass jars of 50 g.

### Principle

The objective of this test is to subject the test sample to extreme (forced) temperature conditions and temperature shocks in order to assess possible stability issues these temperature conditions may lead to in the test sample. Following this nine-day protocol (comprising three cycles of three days each; each cycle consists in subjecting the test sample(s) for 24 hours each at 50° C., 25° C. & 4° C.), any sign of instability suggests probability of potential stability issues. While if the sample is found to be stable without any noticeable issues, in most likely cases the sample is going to be stable under conventional stability protocol. This protocol makes it possible to get a good idea about the stability after only 9 days of time.

### Procedure

About 50 g of the test samples in glass jars are put in triplicate on 50° C. stability chamber for 24 hours. The samples are then transferred to 25° C. chamber for next 24 hrs. Followed by this they are shifted to 4° C. chamber for another 24 hrs. This completes the first cycle, for a total of three cycles. One jar out of three is then analyzed. Different parameters are assessed such as appearance, color, perfume, odor, pH and viscosity. If the product is compliant with the target values, the product is considered as stable after one cycle. The two remaining samples go through a second cycle (24 hours each at 50° C., 25° C. & 4° C.) and then one glass jar is removed and the analysis is repeated. If the product is compliant with the target values, the product is considered as stable after two cycles. Finally, the last glass jar goes through a third cycle. After analysis, if the product is compliant after 3 cycles, it is considered as stable under stressed conditions and the product is also likely to be stable under conventional stability protocol.
All the observations have to be noted down and if any deviation is not acceptable then the product is considered as not stable under stressed conditions and will most probably not be stable under conventional stability protocol as well.
Protocol of Measurement of the Sensorial Effect by a Sensory Panel with Universal Profile:
Location of the test: INDIA
Objectives: To evaluate and compare the sensory properties of two formulas
Experimental procedure: Sequential monadic evaluation in blind randomized presentation
Panel: 17 trained women, 18-35 year old
Evaluation Zone & Time: Product appearance—during application, immediately post application and after 2 minutes of application.

### Protocol of Measurement of the Sensorial Effect by the Formulator in the Lab:

Location of the test: INDIA
Objectives: To evaluate and compare the sensory properties of two formulas
Experimental procedure: Application of 2 known products, one on each front side of the arm
Evaluator: Experienced formulator
Evaluation Zone & Time: Product appearance—during application, immediately post application and after 2 minutes of application.

| | FORMULATION | | | | | | | |
|---|---|---|---|---|---|---|---|---|
| | A | | | | | | | |
| | (invention) | B* | C* | D* | E* | F* | G* | H* |
| pH | 4.03 | — | 4.35 | 4.48 | 4.36 | 4.17 | 4.17 | 4.01 |
| Viscosity (UD) T0 | 50.5 | — | 26.6 | 3 | 43.7 | 66.5 | 46.3 | 34.6 |
| Viscosity (UD) T30 s | 50.1 | — | 26 | 2.8 | 42.7 | 60 | 42.4 | 33.8 |
| Viscosity (UD) T10 min | 48.73 | — | 25.21 | 2.5 | 42.36 | 51.36 | 37.34 | 33.05 |
| Stability comments | ok | 2 phases | ok | 2 phases | ok | Presence of | White | |
| at first sight | | totally | | totally | | few white | aggregates | |
| | | unstable | | unstable | | particles | of polymer | |
| | | and liquid | | and liquid | | on surface | crashing out | |
| Dispersion TiO2 | ok | NO | ok | NO | ok | ok | ok | |
| Noodling | NO | — | NO | — | ok | Yes | Yes, a lot | |
| Texture | ok | — | Light and | — | Sticky and | Very Sticky | Sticky like | Sticky |
| | | | easy to | | drying sensory | unacceptable | a face wash | |
| | | | spread light | | | sensory for | | |
| | | | squeaky feel | | | a leave on | | |
| Stress cycle | Stable | NA | Stable | NA | Stable | Unstable | NA | NA |
| Classical stability | 2 mth Stable | NA | Some | NA | NA | NA | NA | NA |
| | | | crystals | | | | | |
| | | | observed | | | | | |
| | | | at 1 mth | | | | | |
| | | | at 2-8° C. | | | | | |

As is shown above, a number of cellulose-based polymers were tested. However, hydroxypropylmethyl cellulose-based formulations (E and F) resulted in highly sticky formulations and took significantly a longer time to absorb on the skin, which is not appreciated.

HEC-based formulations D and G helped building the viscosity and resulted in non-sticky formulas. However, such HEC formulas mostly resulted in a noodling effect, i.e. the formulation or the polymer comes off with normal rubbing, thus resulting in non-acceptance by the consumers. Moreover, HEC alone does not provide the needed gel strength to hold the titanium dioxide particles suspended in the formulation, as shown by formula D.

As a conclusion, it appears that only formula A according to the invention is stable, shows a dispersion of TiO2, no noodling, and presents the required texture.

Beside, formula A was clinically tested for its efficiency on acne treatment: the results show significant anti-acne performance in reducing non-inflammatory lesions in 2 weeks, on inflammatory lesions in 3 weeks, and on skin sebum level in 1 week.

Finally, a formula A′ according to the invention, similar to formula A according to the invention, was prepared in a similar manner as described above: formula A′ has a similar composition to formula A, except that (i) the 1% dimethicone (5 cst) was substituted by 1% LexFeel™ N5 MB (Inolex), and (ii) the perfume was present in 0.4% (instead of 0.1% for formula A).

### Example 2: Preparation of Compositions According to the Present Invention

Formulas F1 to F4 according to the invention were prepared according to the amounts given in the table below, according to the same protocol as the one of example 1. The amounts are given in % by weight of the total composition.

Viscosity was measured according to the same protocol as the one of example 1.

| | F1 | F2 | F3 | F4 |
|---|---|---|---|---|
| Tetrasodium glutamate | 0.1 | 0.1 | 0.1 | 0.1 |
| diacetate (Dissolvine GL- | | | | |
| 47-S from AkzoNobel) | | | | |
| Dipropylene Glycol | 4 | 4 | 5 | 5 |
| Isododecane | — | — | 1 | 1 |
| Dimethicone (5 cst) | 1 | 1 | 1 | 1 |
| Sodium Hyaluronate | 0.03 | 0.03 | 0.03 | 0.03 |
| Glyceryl stearate (and) | 1 | 1 | 1 | 1 |
| PEG-100 Stearate (Arlacel | | | | |
| 165 from Croda) | | | | |
| Hydroxyethyl cellulose | 0.3 | 0.3 | 0.3 | 0.3 |
| AMMONIUM | 1.5 | 1.5 | 1.5 | 1.5 |
| POLYACRYLOYLDIMETHYL | | | | |
| TAURATE | | | | |
| (Hostacerin ® from | | | | |
| Clariant) | | | | |
| Talc | — | — | 1 | 1 |
| TiO2 (water soluble) | 1.5 | 1 | 1 | 1 |
| Hydroxypropyl starch | — | — | 1 | 1 |
| phosphate (Structure XL) | | | | |
| Salicylic Acid | 1.2 | 1.2 | 1.2 | 1.2 |
| Niacinamide | 2 | 2 | 2 | 2 |
| Zinc gluconate | — | 0.5 | 0.5 | 0.5 |
| Octadecenedioic acid | — | — | 0.5 | 0.5 |
| Piroctone Olamine | — | — | — | 0.5 |
| Menthol Natural | — | 0.02 | — | — |
| Menthol classic | — | | 0.03 | 0.03 |
| Perfume | 0.1 | 0.1 | 0.1 | 0.1 |
| Water | Qsp 100 | Qsp 100 | Qsp 100 | Qsp 100 |
| VISCOSITY T30s (UD) | 38.9 | 35 | 62 | 60 |
| OBSERVATION IN LAB | Very little | | No noodling | |
| (upon preparation) | noodling | | approved by | |
| | | | sensorial | |
| | | | experts | |
| Other comments | Acceptable | Base for oil | | |
| | consistency | control | | |
| | | ingredients | | |

Formulas F1 to F4 according to the invention are stable, show no noodling, and present the required texture.

### Example 3: Preparation of Comparative Compositions

Comparative formulas C1 to C3 were prepared according to the amounts given in the table below, according to the same protocol as the one of example 1. The amounts are given in % by weight of the total composition.

Viscosity was measured according to the same protocol as the one of example 1.

| | C1 | C2 | C3 |
|---|---|---|---|
| Tetrasodium glutamate diacetate | — | 0.1 | 0.1 |
| (Dissolvine GL-47-S from | | | |
| AkzoNobel) | | | |
| Dipropylene Glycol | 3 | 4 | 4 |
| Propylene Glycol | 3 | — | — |
| Coco Betaine | 0.75 | — | — |
| Hydrogenated Castor Oil | 0.75 | — | — |
| Ethanol | 3 | — | — |
| UV FILTERS | YES | NO | NO |
| Dimethicone (5 cst) | — | 1 | 1 |
| Dimethicone (50 cst) | 0.5 | — | — |
| Sodium Hyaluronate | 0.05 | 0.03 | 0.03 |
| Glyceryl stearate (and) PEG-100 | — | 1 | 1 |
| Stearate (Arlacel 165 from Croda) | | | |
| Hydroxyethyl cellulose (HEC) | 1.5 | — | — |
| Xanthan gum | — | 0.25 | — |
| Sepigel 305 (Seppic) | 1 | | — |
| (POLYACRYLAMIDE (and) C13-14 | | | |
| ISOPARAFFIN (and) LAURETH-7 in an | | | |
| inverse emulsion at 40% in | | | |
| isoparaffin/water) | | | |
| Cetyl hydroxyethylcellulose | — | — | 1 |
| Cetearyl Alcohol | — | 1 | — |
| Carbomer (Carbopol Ultrez 30) | — | 1 | — |
| TiO2 (oil soluble) | 0.5 | — | — |
| TiO2 (water soluble) | 0.5 | 1 | 1 |
| Salicylic Acid | 1.2 | 2 | 1.2 |
| Niacinamide | 2 | 2 | 2 |
| Zinc gluconate | — | 0.5 | — |
| Menthol natural | — | 0.02 | — |
| Perfume | 0.1 | 0.1 | 0.1 |
| VISCOSITY t30s (UD) | 74 | 3 | 4.3 |
| OBSERVATION IN LAB | Observation of | No | No thickening, |
| (upon preparation) | noodling effect | thickening | long |
| | due to high HEC | | absorption and |
| | concentration | | unstable |

Xanthan gum could not build the required viscosity as shown for formulation C2, and also resulted in a sticky feeling on skin.

Similarly, cetylhydroxyethylcellulose-based formulations similarly took a longer time to absorb, did not thicken and do not lead to stable formulations (C3).

As a conclusion, it appears that comparative compositions C1 to C3 show noodling, or do not show the required texture, because of no thickening of the composition.

1. A composition comprising, in an aqueous phase:
at least one anti-acne active agent; and
hydroxyethylcellulose and at least poly(2-acrylamido 2-methyl propane sulfonic acid), wherein the weight ratio of active material of poly(2-acrylamido 2-methyl propane sulfonic acid):(hydroxyethylcellulose) ranges from 3:1 to 30:1.

2. The composition according to claim 1, wherein the anti-acne agent is chosen from:
salicylic acid and its derivatives;
niacinamide, niacin, and nicotinic acid esters;
peroxides, including benzoyl peroxide, stabilized hydrogen peroxide and peroxides of organic acids;
metal gluconate;
asiatic acid,
the monoethanolamine salt of 1-hydroxy-4-methyl 6-trimethylpentyl-2-pyridone;
citronellic acid, perillic acid,
glyceryl 2-ethylhexyl ether,
glyceryl caprylate/caprate;
sodium calcium phosphosilicate;
silver-based particles;
hop cone extract (Humulus lupulus) obtained by supercritical CO₂ extraction,
St. John's Wort extract obtained by supercritical CO₂ extraction,
the mixture of extracts of roots of Scutellaria baicalensis, of Paeonia suffruticosa and Glycyrrhiza glabra,
argan tree extract;
bearberry leaf extracts;
10-hydroxy-2-decanoic acid, sodium ursolate, azelaic acid, diiodomethyl p-tolyl sulfone, malachite powder, zinc oxide, octadecenedioic acid; ellagic acid; 2,4,4′-trichloro-2′-hydroxydiphenyl ether, 1-(3′,4′-dichlorophenyl)-3-(4′-chlorophenyl)urea, 3,4,4′-trichlorocarbanilide, 3′,4′,5′-trichlorosalicylanilide, phenoxyethanol, phenoxypropanol, phenoxyisopropanol, hexamidine isethionate, metronidazole and salts thereof, miconazole and salts thereof, itraconazole, terconazole, econazole, ketoconazole, saperconazole, fluconazole, clotrimazole, butoconazole, oxiconazole, sulfaconazole, sulconazole, terbinafine, ciclopirox, ciclopiroxolamine, undecylenic acid and salts thereof, 3-hydroxybenzoic acid, 4-hydroxybenzoic acid, phytic acid, N-acetyl-L-cysteine, lipoic acid, arachidonic acid, resorcinol, 3,4,4′-trichlorocarbanalide, octoxyglycerine or octoglycerine, octanoylglycine, caprylyl glycol, 10-hydroxy-2-decanoic acid, dichlorophenylimidazoldioxolane, iodopropynyl butylcarbamate, 3,7,11-trimethyldodeca-2,5,10-trienol or farnesol, phytosphingosines; quaternary ammonium salts, for instance cetyltrimethylammonium salts and cetylpyridinium salts, and mixtures thereof.

3. The composition according to claim 1, wherein the anti-acne agent is present in the composition of the present invention in an amount ranging from 0.01% to 20% by weight relative to the total weight of the composition.

4. The composition according to claim 1, wherein the aqueous phase is present in an amount ranging from 10% to 99% by weight of the total weight of the composition.

5. The composition according to claim 1, wherein the aqueous phase comprises water, and optionally at least one polyol and/or at least one organic solvent miscible with water.

6. The composition according to claim 1, wherein the aqueous phase further comprises sodium hyaluronate.

7. The composition according to claim 1, wherein hydroxyethylcellulose is present in amounts of active material ranging from 0.01 to 20% by weight relative to the total weight of the composition.

8. The composition according to claim 1, wherein the poly(2-acrylamido 2-methyl propane sulfonic acid) is crosslinked or non-crosslinked; and/or is partially or completely neutralized with an inorganic base or an organic base, and mixtures of these compounds.

9. The composition according to claim 1, wherein the poly(2-acrylamido 2-methyl propane sulfonic acid) is partially neutralized with ammonia and highly cross-linked.

10. The composition according to claim 1, wherein the poly(2-acrylamido 2-methyl propane sulfonic acid) is present in amounts of active material ranging from 0.01 to 20% by weight relative to the total weight of the composition.

11. The composition according to claim 1, wherein the weight ratio of active material of poly(2-acrylamido 2-methyl propane sulfonic acid):(hydroxyethylcellulose) ranges from 4:1 to 25:1.

12. The composition according to claim 1, which further comprises at least one surfactant and/or a fatty phase and/or at least one crosslinked starch.

13. The composition according to claim 1, which has a pH of less than 4.5.

14. The composition according to claim 1, which comprises an emollient.

15. A non-therapeutic method for treating a keratin material comprising the step of applying the composition according to claim 1 to the keratin material.

16. A method for treating acne, comprising the step of applying the composition according to claim 1 onto skin.

17. The composition according to claim 2, wherein the anti-acne agent is present in the composition of the present invention in an amount ranging from 0.01% to 20% by weight, relative to the total weight of the composition.

18. The composition according to claim 2, wherein the aqueous phase is present in an amount ranging from 10% to 99% by weight of the total weight of the composition.

19. The composition according to claim 3, wherein the aqueous phase is present in an amount ranging from 10% to 99% by weight of the total weight of the composition.

20. The composition according to claim 2, wherein the aqueous phase comprises water, and optionally at least one polyol and/or at least one organic solvent miscible with water.
